SEQUENCE LISTING (19 sequences)

SEQ ID NO: 1 - DNA, 19 bp, Artificial Sequence
  Description of Artificial Sequence UTR-L2 forward primer
cgnctagccatggcgttag

SEQ ID NO: 2 - DNA, 19 bp, Artificial Sequence
  Description of Artificial Sequence C-A-2 forward primer
gagagccatagtggtctgc

SEQ ID NO: 3 - DNA, 18 bp, Artificial Sequence
  Description of Artificial Sequence UTR-R2 reverse primer
caggcagtaccacaaggc

SEQ ID NO: 4 - DNA, 19 bp, Artificial Sequence
  Description of Artificial Sequence UTR-L1 forward primer
ggaactwctgtcttcacgc

SEQ ID NO: 5 - DNA, 17 bp, Artificial Sequence
  Description of Artificial Sequence UTR-R1 reverse primer
acggtctacgagacctc

SEQ ID NO: 6 - DNA, 17 bp, Artificial Sequence
  Description of Artificial Sequence Spec-1 primer
ccgctcaatgcctggag

SEQ ID NO: 7 - DNA, 22 bp, Artificial Sequence
  Description of Artificial Sequence Spec-2 primer
ctttcttggataaacccactct

SEQ ID NO: 8 - DNA, 17 bp, Artificial Sequence
  Description of Artificial Sequence Spec-3 primer
gtgcccccgcgagatca

SEQ ID NO: 9 - DNA, 22 bp, Artificial Sequence
  Description of Artificial Sequence Spec-4-Thy primer
ccatggcgttagtatgagtttt

SEQ ID NO: 10 - DNA, 22 bp, Artificial Sequence
  Description of Artificial Sequence Spec-4-Ad primer
ccatggcgttagtatgagtatt

SEQ ID NO: 11 - DNA, 24 bp, Artificial Sequence
  Description of Artificial Sequence L1 probe
cgcnacccaacnctactnggctag

SEQ ID NO: 12 - DNA, 38 bp, Artificial Sequence
  Description of Artificial Sequence MBP-LR-1 forward primer
caccttcaccctcagaaggngccgctcaatgcctggag

SEQ ID NO: 13 - DNA, 40 bp, Artificial Sequence
  Description of Combined DNA/RNA Molecule MBP-LR-ALL forward primer
caccttcaccctcagaaggngcguctagccatggcgttag

SEQ ID NO: 14 - DNA, 20 bp, Artificial Sequence
  Description of Artificial Sequence Spec-4-Thy 4a primer
atggcgttagtatgagtgtt

SEQ ID NO: 15 - DNA, 21 bp, Artificial Sequence
  Description of Artificial Sequence Spec-4-Thy 4b primer
catggcgttagtatgagtgtt

SEQ ID NO: 16 - DNA, 22 bp, Artificial Sequence
  Description of Artificial Sequence Spec-4-Thy 4c primer
ccatggcgttagtatgagtgtt

SEQ ID NO: 17 - DNA, 22 bp, Artificial Sequence
  Description of Artificial Sequence Spec-4-Thy 4-Ino
ccatggcgttagtatgagtntt

SEQ ID NO: 18 - DNA, 227 bp, Hepatitis C virus
aaagcgtctagccatggcgttagtatgagtgtcgtgcagcctccaggaccccccctcccgggagagccatagtggtctgcggaaccggtgagtacaccggaattgccaggacgaccgggtcctttcttggataaacccgctcaatgcctggagatttgggcgtgcccccgcaagactgctagccgagtagtgttgggtcgcgaaaggccttgtggtactgcctgata

SEQ ID NO: 19 - DNA, 19 bp, Artificial Sequence
  Description of Artificial Sequence UTR-L1 forward primer
ggaacttctgtcttcacgc

(19) United States
(12) Patent Application Publication
(10) Pub. No.: US 2009/0130649 A1
(43) Pub. Date: May 21, 2009

(54) Methods for Genotyping HVC

(75) Inventors: Richard Cross, Southampton (GB); William Malcolm Charles Rosenberg, Southampton (GB)

Correspondence Address: LAHIVE & COCKFIELD, LLP;FLOOR 30, SUITE 3000, ONE POST OFFICE SQUARE, BOSTON, MA, 02109, US

(73) Assignee: IQUR LIMITED, Southampton (GB)

(21) Appl. No.: 11/666,176

(22) PCT Filed: Oct. 21, 2005
(86) PCT No.: PCT/GB05/04097

§ 371 Date: Jan. 9, 2008

(30) Foreign Application Priority Data
Oct. 22, 2004 (GB) 0423552.9

(51) Int. Cl. C12Q 1/70; C07H 21/04

(52) U.S. Cl. 435/5; 536/24.33

(57) ABSTRACT

The present invention relates to methods for differentiating Hepatitis C virus (HCV) group A genotypes from HCV group B genotypes. The invention finds application in determining prognosis, and in the selection of treatment regimes, for patients infected with HCV.

This invention relates to methods for differentiating Hepatitis C virus (HCV) group A genotypes from HCV group B genotypes. The invention therefore finds application in determining prognosis, and in the selection of treatment regimes, for patients infected with HCV.

The hepatitis C virus is a positively stranded RNA virus that exists in six genetically distinct genotypes. These are designated Type 1, 2, 3, 4, 5 and 6, and their full length genomes have been reported (Genbank/EMBL accession numbers Type 1a: AF011753; Type 1b: AF054250; Type 2a: D00944; Type 2b: D10988; Type 2c: D50409; Type 3a: AF046866; Type 3b: D49374; Type 4a: Y11604; Type 5a: AF064490; Type 6: D84262). Viruses in each genotype exist as differing “quasispecies” that exhibit minor genetic differences. In the UK and USA, the vast majority of infected individuals are infected with genotype 1, 2 or 3 HCV. In the UK, genotype 4 is identified at low prevalence in some immigrant populations, but is common in the Middle East and Africa. Genotypes 5 and 6 are rarely encountered in the UK but are more common in the Far East. HCV infection affects approximately 0.5% of the population in the United Kingdom, 1.8% of the population in the USA and 3% of the population of the world. In over 85% of infected people, HCV causes a lifelong infection characterised by chronic hepatitis that varies in severity between individuals.

The only effective therapy that is currently available is interferon alpha (IFN-α) used alone in modified form conjugated with polyethyleneglycol (PEG) or in conjunction with ribavirin (“combination therapy”). Randomised controlled trials have shown that combination therapy is highly effective in the treatment of HCV infection in patients previously treated with interferon alone and in patients never previously treated with interferon (Davis et al, NEJM, (1998), 339(21): 1493-99; Poynard et al, Lancet (1998) 352(9138): 1426-32).

Subgroup analysis has revealed that by far the major determinant of prognosis and response to treatment is HCV genotype (Poynard et al, Lancet, 1998, 352(9138): 1426-32). For patients with genotype 2 or 3 infections treated for six months, response rates to treatment for six months with reduced dose ribavirin exceed those seen in patients with genotype 1, 4, 5 or 6 infections for one year. In mixed genotype infections, genotype 1, 4, 5 or 6 predominate.

In view of the difference in treatment terms for HCV genotypes 2 and 3, compared to HCV genotypes 1, 4, 5 and 6, these genotypes have been classified into the following groups:

HCV genotype group A —HCV genotypes 1, 4, 5 and 6
HCV genotype group B—HCV genotypes 2 and 3

Combination therapy is expensive (in the UK, it costs approximately £8,000 for one year's treatment). Thus, the identification of patients with group B infections allows treatment to be stopped after 6 months with a consequent cost saving, and the avoidance of unnecessary adverse effects of the drugs associated with further treatment. In addition, the most serious outcome of HCV infection is hepatocellular carcinoma. It is therefore desirable to be able to identify with which genotype group of HCV a patient is infected.

Known HCV genotyping methods rely on agarose gel electrophoresis of restriction endonuclease digested polymerase chain reaction amplification products (RFLP), detection of HCV using specific antibodies, sequence analysis by direct sequencing, or by Bayer Versant LiPA Assay. Enzymatic digestion can take a relatively long time to reach completion making the RFLP method time consuming. It also involves several different stages, making it unamenable to automation or rapid throughput. In addition, inaccurate genotyping can occur owing to (i) the fact that restriction endonucleases do not cut with 100% efficiency, and (ii) single base pair mutations in quasispecies may result in failure of digestion of the PCR products. The LiPA assay is a commercially available kit for HCV genotyping, but is labour intensive, time consuming, not amenable to high throughput and can be difficult to interpret. Sequencing is dependent on specialist equipment and trained operators and is also time consuming.

U.S. Pat. No. 5,851,759 discloses methods of genotyping HCV in which HCV RNA is isolated and cDNA is synthesised from this RNA. The cDNA is then subjected to PCR using primers which flank the E1 region of the HCV genome. The products of PCR are analysed using the Heteroduplex Tracking Assay (HTA). The E1 region was selected for analysis because it is considered to be the most heterogeneous region of the HCV genome. Thus, amplification of this region is likely to give a different product for each HCV strain in a sample. These products can then be analysed to indicate the strains that are present in the sample.

WO 01/46469 discloses the use of a non-specific primer to amplify the 5′ noncoding region of any HCV present in a specimen so that the sequence thereof can be determined.

Bukh J et al, Proc. Natl. Acad. Sci. USA, 1992, 89:4942-4946 discloses a sequence analysis of the 5′ noncoding region of various HCV isolates. The results indicate that there are regions of heterogeneity between the isolates tested. The authors were not able to say whether the heterogeneity observed was between different subgroups. As a result, the authors concluded that the 5′ noncoding region of HCV is not a region which should be used in the identification of HCV.

WO 03/057915 discloses a method for differentiating HCV-1 from HCV-2 and HCV-3. However, the method does not provide a definitive answer as to whether the HCV is a genotype group A or group B.

In view of the different treatment terms for treating HCV genotype group A and HCV genotype group B, there exists a need to be able to distinguish between these HCV genotype groups.

According to a first aspect of the present invention, there is provided a method of determining whether HCV that is present in a sample belongs to HCV genotype group A or HCV genotype group B, comprising:
(a1) (i) subjecting the sample to a first amplification reaction using at least one primer which anneals specifically to the 5′ noncoding region (5′ NCR) of the HCV-2 genome; and
   (ii) subjecting the sample to a second amplification reaction using at least one primer which anneals specifically to the 5′ NCR of the HCV-3 genome; or
(a2) subjecting the sample to an amplification reaction using at least one primer which anneals specifically to the 5′ NCR of the HCV-2 genome, and at least one primer which anneals specifically to the 5′ NCR of the HCV-3 genome; and
(b) detecting the products of the amplification reaction(s),
   wherein, if a product is detected in step (a1) or (a2), the HCV is in group B and, if no product is detected in step (a1) or (a2), the HCV is in group A.

In the method of the first aspect of the invention, if HCV genotype 2 or 3 is detected, then HCV genotype group B is present. In contrast, if HCV genotype 2 or 3 is not detected, then HCV genotype group A is present.

The method of the first aspect may further comprise:
(c) subjecting the sample to an amplification reaction using at least one primer which anneals specifically to the 5′ noncoding region (5′ NCR) of the genomes of HCV-1, HCV-5 and HCV-6; and/or
(d) subjecting the sample to an amplification reaction using at least one primer which anneals specifically to the 5′ NCR of the HCV-4 genome; and
(e) detecting the products of the amplification reactions,
   wherein, if a product is detected in step (c) and/or (d), the HCV is in HCV group A and, if no product is detected in step (c) and/or (d), the HCV is in HCV group B.

If a product is detected in step (c) then the HCV is HCV genotype 1, 5 or 6, and if a product is detected in step (d) then the HCV is HCV genotype 4.

In a second aspect, the present invention provides a method for determining whether HCV that is present in a sample belongs to HCV genotype group A or HCV genotype group B, comprising:
(a1) (i) subjecting the sample to a first amplification reaction using at least one primer which anneals specifically to the 5′ noncoding region (5′ NCR) of the genomes of HCV-1, HCV-5 and HCV-6; and
   (ii) subjecting the sample to a second amplification reaction using at least one primer which anneals specifically to the 5′ NCR of the HCV-4 genome; or
(a2) subjecting the sample to an amplification reaction using at least one primer which anneals specifically to the 5′ NCR of the genomes of HCV-1, HCV-5 and HCV-6, and at least one primer which anneals specifically to the 5′ NCR of the HCV-4 genome; and
(b) detecting the products of the amplification reactions,
wherein, if a product is detected in step (a1) or step (a2), the HCV is in group A and, if no product is detected in step (a1) or step (a2), the HCV is in group B.

In the method of the second aspect of the invention, if HCV genotype 1, 4, 5 or 6 is detected, then HCV genotype group A is present. In contrast, if HCV genotype 1, 4, 5 or 6 is not detected, then HCV genotype group B is present. If a product is detected in step (a1)(i) then the HCV is HCV genotype 1, 5 or 6, and if a product is detected in step (a1)(ii) then the HCV is HCV genotype 4.

The method of the second aspect may further comprise:
(c) subjecting the sample to an amplification reaction using at least one primer which anneals specifically to the 5′ noncoding region (5′ NCR) of the HCV-2 genome; and/or
(d) subjecting the sample to an amplification reaction using at least one primer which anneals specifically to the 5′ NCR of the HCV-3 genome; and
(e) detecting the products of the amplification reactions,
   wherein, if a product is detected in step (c) and/or (d), the HCV is in HCV group B and, if no product is detected in step (c) and/or (d), the HCV is in HCV group A.

If a product is detected in step (c) then the HCV is HCV genotype 2, and if a product is detected in step (d) then HCV is HCV genotype 3.

In a third aspect, the present invention provides a method of detecting HCV genotype 1, 5 or 6 (HCV-1, HCV-5 or HCV-6), HCV genotype 2 (HCV-2), HCV genotype 3 (HCV-3), or HCV genotype 4 (HCV-4) in a sample, comprising:
(a1) (i) subjecting the sample to an amplification reaction using at least one primer which anneals specifically to the 5′ noncoding region (5′ NCR) of the genomes of HCV-1, HCV-5 and HCV-6; and
   (ii) subjecting the sample to an amplification reaction using at least one primer which anneals specifically to the 5′ NCR of the HCV-2 genome; and
   (iii) subjecting the sample to an amplification reaction using at least one primer which anneals specifically to the 5′ NCR of the HCV-3 genome; and
   (iv) subjecting the sample to an amplification reaction using at least one primer which anneals specifically to the 5′ NCR of the HCV-4 genome; or
(a2) subjecting the sample to an amplification reaction using at least one primer which anneals specifically to the 5′ NCR of the genomes of HCV-1, HCV-5 and HCV-6, at least one primer which anneals specifically to the 5′ NCR of the HCV-2 genome; at least one primer which anneals specifically to the 5′ NCR of the HCV-3 genome; and at least one primer which anneals specifically to the 5′ NCR of the HCV-4 genome; and
(c) detecting the products of the amplification reactions.

Accordingly, the methods of the first, second and third aspects of the present invention determine whether the HCV belongs to HCV genotype group A or HCV genotype group B, and also allow HCV genotypes 1, 5, 6, HCV genotype 2, HCV genotype 3, and HCV genotype 4 to be distinguished from each other. Therefore, the methods of the first, second and third aspects of the present invention provide suitable information to make a judgement on which treatment regime should be followed as determined by the genotype of the HCV infection without the need for lengthy, expensive and unnecessary detailed genotyping and subtyping.

The HCV genome consists of 5′ and 3′ noncoding (or untranslated) regions (NCR) that flank a single long open reading frame (ORF). This ORF encodes for three structural proteins at the amino-terminal end and for six non-structural (NS) proteins at the carboxyl-terminal end. The structural proteins are represented from the nucleocapsid (core; C) proteins and two glycoproteins, envelope 1 (E1) and envelope 2 (E2). The non-structural proteins are named NS2, P7, NS3, NS4a, NS4b, NS5a, NS5b. The 5′ NCR is the most highly conserved part of the HCV genome, whereas the sequence of the two envelope proteins (E1 and E2) is highly variable among different HCV isolates. The present invention lies in the surprising finding that parts of the 5′ NCR are conserved for each of HCV genotypes 1, 2, 3, 4, 5 and 6. Accordingly, primers for amplification reactions can be designed which anneal specifically to those parts of the 5′ NCR that are conserved for the respective HCV genotypes.

The amplification reaction may be PCR (see for example U.S. Pat. Nos. 4,683,195 and 4,683,202, and Innis et al, editors, PCR Protocols, (Academic Press, New York, 1989; Sambrook et al, Molecular Cloning, Second Edition, (Cold Spring Harbour Laboratory, New York 1989)). PCR will generally be used when HCV RNA has been isolated and converted, preferably by reverse transcription, to cDNA. Preferably, PCR is carried out using Taq DNA polymerase, e.g. Amplitaq™ (Perkin-Elmer, Norwalk, Conn.). Taq polymerase can also be obtained from MBI Fermentas, Perkin Elmer, Boehringer Mannheim and Beckman Instruments. An equivalent, preferably thermostable, DNA polymerase may also be used in the method of the present invention, such as Tfl (Thermus flavus) polymerase (Gut et al, Virol. Methods 77(1): 37-46 (1999)).

Alternatively, the amplification reaction may be RT-PCR (Yajima et al, Clin. Chem., 44(12): 2441-2445 (1998); Martell et al, J. Clin. Microbiol., 37(2): 327-332 (1999); Gut et al, Virol. Methods 77(1): 37-46 (1999); Predhomme et al, Leukemia, 13(6): 957-964 (1999)), in which the HCV RNA is reverse transcribed into cDNA which is then subjected to PCR amplification.

The reverse transcription of HCV RNA to cDNA and the subsequent PCR amplification reaction may be performed in one step. In this way, the amplification reaction of the methods of the present invention may comprise:
a) raising the temperature of a reaction mix comprising HCV RNA, reverse transcriptase, DNA polymerase and primers, to activate the reverse transcriptase; and
b) raising the temperature further to inactivate the reverse transcriptase and to activate the DNA polymerase.

In this way, the amplification reaction step is more efficient because it requires less hands-on time for the operator. As a result of this, the risk of contamination of the sample is reduced.

Other components, such as buffers and enzymes may be incorporated into the reaction mix for the one-step amplification reaction. The components of the reaction mix other than the HCV RNA and primers are commercially available such as, for example, from Qiagen (QuantiTect Probe RT-PCR Kit).

The DNA polymerase may be Taq DNA polymerase. The Taq DNA polymerase may be a “Hot start” Taq DNA polymerase. It may be HotStarTaq DNA polymerase.

The temperature in step a) may be raised to a temperature falling within the range of approximately 40° C. to approximately 60° C., and preferably falling within the range of approximately 45° C. to approximately 55° C. The temperature in step a) may be raised to approximately 50° C. The temperature in step b) may be raised to a temperature falling within a range of approximately 80° C. to approximately 110° C., and preferably falling within a range of approximately 90° C. to approximately 100° C. The temperature in step b) may be raised to approximately 95° C.

As is well-known, PCR involves the extraction and denaturation (preferably by heat) of a sample of DNA (or RNA). A molar excess of oligonucleotide primers is added, along with a polymerase, which may be heat-stable, and dNTPs for forming the amplified sequence. The oligonucleotide primers are designed to hybridise to opposite ends of the sequence which it is desired be amplified. In the first amplification round, the polymerase replicates the DNA to produce two “long products,” which begin with the respective primers. The total DNA, which includes the two long products and the two original strands, is then denatured and a second round of polymerisation is carried out (for example, by lowering the temperature). The result of the second round is the two original strands, the two long products from the first round, two new long products (produced from the original strands), and two “short products” produced from the long products. These short products have the sequence of the target sequence (sense or antisense) with a primer at each end. For each additional amplification round, the number of short products grows exponentially, each round producing two additional long products and a number of short products equal to the sum of the long and short products remaining at the end of the previous round.

Oligonucleotide primers can be synthesised by a number of approaches, e.g. Ozaki et al, Nuc. Acids Res. 20: 5205-5214 (1992); Agrawal et al, Nuc. Acids Res. 18: 5419-5423 (1990) or the like. Conveniently, the oligonucleotide probes are synthesised on an automated DNA synthesiser, e.g. an Applied Biosystems, Inc, Foster City, Calif. model 392 or 394 DNA/RNA synthesiser using standard chemistries such as phosphoramidite chemistry (Beaucage and Iyer, Tetrahedron 48: 2223-2311 (1992), U.S. Pat. Nos. 4,980,460, 4,725,677, 4,415,732, 4,458,066 and 4,973,679). Alternative chemistries, including non-natural backbone groups such as phosphorothioate and phosphoramidate, may also be employed, provided that the hybridisation efficiencies of the resulting oligonucleotides are not adversely affected. The precise length and sequence of the DNA primers will depend on the target polynucleotide to be amplified. Preferably, the length of the DNA primers is in the range 10 to 60 nucleotides and more preferably in the range 15 to 30 or 25 nucleotides.

The methods of the first, second and third aspects may also comprise subjecting the sample to a preliminary amplification reaction to detect whether any HCV genotype is present in the sample using primers which anneal to a region of the 5′ NCR which is conserved between all HCV genotypes. The primers that anneal to a region of the 5′ NCR which is conserved between all HCV genotypes may be (where nucleic acid positions are referred to herein, they are from the consensus sequence M67463 from Fields' Virology):

Forward

(SEQ ID NO: 1)

i) 5′ CGI CTA GCC ATG GCG TTA G 3′;
(UTR-L2) (position 76-94) or

(SEQ ID NO: 2)

ii) 5′ GAG AGC CAT AGT GGT CTG C 3′
(C-A-2) (position 133-151)
Reverse:

(SEQ ID NO: 3)

5′ CAG GCA GTA CCA CAA GGC 3′
(UTR-R2) (position 295-278)
Where I = deoxyinosine

The methods of the first, second and third aspects of the present invention may also comprise a further preliminary step in which HCV material is isolated from the sample. In this step, the sample is subjected to a preliminary amplification reaction using primers universal for all HCV genotypes. This step therefore enables the method of the invention to be applied to a sample that includes HCV material. Suitable primers for this are:

Forward:

(SEQ ID NO: 4)

5′ GGA ACT WCT GTC TTC ACG C 3′
(UTR-L1) (position 51-69)
Reverse:

(SEQ ID NO: 5)

5′ ACG GTC TAC GAG ACC TC 3′
(UTR-R1) (position 336-320)
Where W = adenine or thymine.

Detection of the products of amplification may be carried out using the well-known technique of agarose gel electrophoresis. If HCV genotypes 2 or 3 (HCV genotype group B) or HCV genotypes 1, 4, 5 or 6 (HCV genotype group A) are present in the sample, the amplification reaction produces a product or products (of known size) which can be detected on the agarose gel.

A primer specific for HCV-1, 5 and 6 suitable for use in the present invention may be:

(SEQ ID NO: 6)

5′ CCG CTC AAT GCC TGG AG 3′
(Spec-1) (position 208 to 224)

An HCV-2 specific primer suitable for use in the present invention may be an oligonucleotide that hybridises specifically to the 5′ NCR of the HCV-2 genome, wherein the oligonucleotide includes the sequence 5′ACTCT 3′, is 8-30 nucleotides long and has no more than 5 nucleotides 3′ of 5′ACTCT 3′. The oligonucleotide may be 15, 16, 17, 18, 19, 20, 21, 22, 23, 24 or 25 nucleotides long. The HCV-2 specific primer may be the oligonucleotide:

(SEQ ID NO: 7)

5′ CTT TCT TGG ATA AAC CCA CTC T 3′
(Spec-2)(position 193-214)

An HCV-3 specific primer suitable for use in the present invention may be an oligonucleotide that hybridises specifically to the 5′ NCR of the HCV-3 genome, wherein the oligonucleotide includes the sequence 5′ TCA 3′, is 8-30 nucleotides long and has no more than 5 nucleotides 3′ of 5′TCA 3′. The oligonucleotide may be 15, 16, 17, 18, 19, 20, 21, 22, 23, 24 or 25 nucleotides long. The HCV-3 specific primer may be the oligonucleotide:

(SEQ ID NO: 8)

5′ GTG CCC CCG CGA GAT CA 3′ (Spec-3).
(position 233-249)

An HCV-4 specific primer suitable for use in the present invention may be an oligonucleotide that hybridises specifically to the 5′ NCR of the HCV-4 genome, wherein the oligonucleotide includes the sequence 5′TWTT 3′, wherein W is adenine or thymine, is 8-30 nucleotides long and has no more than 5 nucleotides 3′ of 5′TWTT 3′. The oligonucleotide may be 15, 16, 17, 18, 19, 20, 21, 22, 23, 24 or 25 nucleotides long. The HCV-4 specific primer may be an oligonucleotide selected from:

(SEQ ID NO: 9)

5′ CCA TGG CGT TAG TAT GAG TTT T 3′ (Spec-4-Thy)
(position 83-104)

(SEQ ID NO: 10)

5′ CCA TGG CGT TAG TAT GAG TAT T 3′ (Spec-4-Ad)

The above primers may be used together with any universal HCV primer. One example is:

(SEQ ID NO: 3)

Reverse: 5′ CAG GCA GTA CCA CAA GGC 3′ (UTR-R2)

The invention also provides the Spec-1, Spec-2, Spec-3, Spec-4-Thy and Spec-4-Ad primers alone and in combination with any universal HCV primer, such as UTR-R2.

In a yet further aspect, the present invention provides a method of detecting HCV genotype 2 (HCV-2) in a sample, comprising:
a) subjecting the sample to an amplification reaction using at least one primer which anneals specifically to the 5′ NCR of the HCV-2 genome; and
b) detecting the product of the amplification reaction. Spec-2 is a suitable HCV-2 specific primer useful in this method.

In a yet further aspect, the present invention provides a method of detecting HCV genotype 3 (HCV-3) in a sample, comprising:
a) subjecting the sample to an amplification reaction using at least one primer which anneals specifically to the 5′ NCR of the HCV-3 genome; and
b) detecting the product of the amplification reaction. Spec-3 is a suitable HCV-3 specific primer useful in this method.

HCV genotype 4 is highly prevalent in the Middle East and Africa, exceeding 25% of the population in Egypt. Trials suggest that genotype 4 responds to treatment in a manner more akin to genotype 1 than genotype 2 and 3 and thus patients with genotype 4 should receive 1 year and full dose Ribavirin. However, emerging evidence suggests that genotype 4 may respond in a manner intermediate between genotype 1 and genotype 2 and 3. Thus, the detection of genotype 4 specifically in order to offer an intermediate treatment regime would be useful. In view of this, in a further aspect, the present invention provides a method of detecting HCV genotype 4 (HCV-4) in a sample, comprising:
a) subjecting the sample to an amplification reaction using at least one primer which anneals specifically to the 5′ NCR of the HCV-4 genome; and
b) detecting the product of the amplification reaction. Spec-4-Thy and Spec-4-Ad are suitable HCV-4 specific primers useful in this method.

The amplification reactions of the methods of the present invention may be performed simultaneously.

As an alternative to agarose gel electrophoresis, detection of the products of the, or each, amplification reaction may alternatively be carried out using fluorescent analysis. If an HCV genotype is present, amplification occurs and this is detected by hybridisation of a quenched fluorescently labelled probe. With successive rounds of amplification, an increasing amount of the fluorescent probe becomes incorporated into PCR products and the quencher is liberated resulting in an increasing amount of fluorescence being detectable on the reaction vessels. Such fluorescence detection makes this approach ideally suited to automation and high throughput processes.

One well-known fluorescent technique is the so-called “TaqMan” method (Holland, et al, Proc. Nat. Acad. Sci, 88: 7276-80, (1985)). This method uses an oligonucleotide probe complementary to a segment of the target polynucleotide, i.e. between the sequence defined by the PCR primers, which probe is labelled with two fluorescent molecules or fluorophores. The emission spectrum of one of the molecules overlaps with the excitation spectrum of the other and, as a result, the emission of the first fluorophore (the “reporter”) is quenched by the second (the “quencher”) when they are in sufficient proximity.

The oligonucleotide probe is hybridised to the target polynucleotide downstream from a primer for a polymerase having a 5′-3′ exonuclease activity. As the polymerase extends the primer as part of the PCR reaction, the oligonucleotide is digested, releasing one of the “quencher” and “reporter” molecules and causing the distance between these molecules to become such that the fluorescent emission is no longer quenched by energy transfer. Thus, a fluorescent signal is generated, providing real-time monitoring of amplification.

The oligonucleotide probe may be made in the same way as the oligonucleotide primers mentioned above and may have the same variations in backbone and so on, providing that hybridisation to the target polynucleotide is not compromised and/or cleavage efficiency of the exonuclease are not adversely affected. Preferably, the length of the oligonucleotide probe is in the range of 10 to 60 nucleotides, and more preferably 18 to 30 nucleotides. The precise length and sequence of the oligonucleotide probe depends at least in part on the nature of the target polynucleotide to which it binds and will be varied to achieve appropriate annealing and melting properties for a particular target polynucleotide. In addition, the binding location of the probe may be varied according to the nature of the target polynucleotide.

Preferred primers for this reaction are UTR-R2 and Spec-1, Spec-2, Spec-3, Spec-4-Thy or Spec-4-Ad mentioned above. A preferred probe anneals to a region of the 5′ NCR which is conserved between all HCV genotypes. One such probe has the following sequence:

(SEQ ID NO: 11)

5′ FCG CIA CCC AAC ICT ACT IGG CTA GT 3′ (L1)
(position (271-250)
where F = 6-FAM,3′-T + TAMRA

Where the reverse transcription of HCV RNA to cDNA and the subsequent PCR amplification reaction are performed in one step, the TaqMan probe may be included in the reaction mix. Therefore TaqMan PCR occurs when raising the temperature to inactivate the reverse transcriptase and to activate the Taq DNA polymerase.

Detection by fluorescent analysis may further comprise determining the HCV RNA concentration (viral load) in the sample. The detection by fluorescent analysis may further comprise (i) detecting the amplification reaction cycle at which the change in fluorescence exceeds a detection threshold; and (ii) determining the RNA concentration by comparing the amplification reaction cycle threshold with known values.

The quantity of starting template RNA determines the quantity of cDNA produced in a reverse transcriptase reaction. The quantity of cDNA in the sample then determines the point at which the PCR product in the TaqMan reaction exceeds a detection threshold. The PCR cycle at which this occurs is the cycle threshold (CT). By performing TaqMan PCR with a series of standard RNA samples of known concentration, it is possible to construct a “standard curve” by plotting the quantity of RNA template per ml in the sample against the CT. From this curve it is possible to estimate the concentration of RNA in a sample by determining the relation to the CT.

A further alternative method of detecting the amplification products is using one or more molecular beacon probes. These are PCR primers comprising a sequence attached to a hairpin loop sequence which contains both a fluorescent label and a quencher molecule. The hairpin brings the fluorescent molecule in opposition to the quencher so that the primer does not fluoresce under normal conditions. As PCR results in copying of the probe sequence, the hair-pin opens and the fluorescent beacon and quencher become separated. The resulting PCR product fluoresces.

In a preferred embodiment, the following PCR primers are used:

Forward: MBP-LR-1

(SEQ ID NO: 12)

5′ FCA CCT TCA CCC TCA GAA GGM GCC GCT CAA TGC CTG
GAG 3′
(F = FAM; M = MeREDdU and U = Uracil)

### Reverse: UTR-R2

MBP-LR-1 is a forward HCV genotype 1, 5, or 6 specific primer and, as mentioned above, UTR-R2 is a universal reverse primer. Any universal HCV primer can be used together with MBP-LR-1.

If it is desired to detect the presence of other HCV genotypes in the sample, an additional amplification can be carried out using at least one molecular beacon probe which is universal for all HCV genotypes. A suitable probe in this regard is MBP-LR-ALL which is a forward primer and has the following sequence:

(SEQ ID NO: 13)

5′ FCACCTTCACCCTCAGAAGGMGCGUCTAGCCATGGCGTTAG 3′
(F = FAM; M = MeREDdU and U = Uracil)

MBP-LR-ALL can be used together with any universal reverse primer; one suitable example is UTR-R2.

Furthermore, molecular beacon probes can be designed that are forward primers specific to HCV genotype 2, HCV genotype 3 or HCV genotype 4. Such molecular beacon probes can be used with any universal reverse primer, such as UTR-R2.

Fluorochromes that emit light at differing wavelengths can be incorporated into the different primers so that products amplified with different primers can be distinguished in the same reaction vessel. Alternative hairpin sequences on the beacon probes can easily be designed by the skilled person.

Alternatively, the amplification products can be detected using DNA hybridisation employing enzyme linked methods to confirm hybridisation, such as using horse radish peroxidase on sequence specific probes and an appropriate substrate.

According to a further aspect of the present invention, there is provided a kit for determining whether HCV that is present in a sample belongs to HCV genotype group A or HCV genotype group B, comprising:
(a) at least one primer which anneals specifically to the 5′ NCR of the HCV genotype 2 (HCV-2) genome; and
(b) at least one primer which anneals specifically to the 5′ NCR of the HCV genotype 3 (HCV-3) genome.

The kit may further comprise:
(c) at least one primer which anneals specifically to the 5′ NCR of the HCV genotype 4 (HCV-4) genome; and
(d) at least one primer which anneals specifically to the 5′ NCR of the genomes of HCV genotypes 1, 5 and 6 (HCV-1, HCV-5 and HCV-6).

A further aspect of the invention provides a kit for determining whether HCV that is present in a sample belongs to HCV genotype group A or HCV genotype group B, comprising:
(a) at least one primer which anneals specifically to the 5′ NCR of the genomes of HCV genotype 1, 5 and 6 (HCV-1, HCV-5 and HCV-6); and
(b) at least one primer which anneals specifically to the 5′ NCR of the HCV genotype 4 (HCV-4) genome.

A further aspect of the invention provides a kit for detecting HCV genotype 2 (HCV-2) in a sample, comprising:
(a) at least one primer which anneals specifically to the 5′ NCR of the HCV genotype 2 (HCV-2) genome.

A further aspect of the invention provides a kit for detecting HCV genotype 3 (HCV-3) in a sample, comprising:
(a) at least one primer which anneals specifically to the 5′ NCR of the HCV genotype 3 (HCV-3) genome.

A further aspect of the invention provides a kit for detecting HCV genotype 4 (HCV-4) in a sample, comprising:
(a) at least one primer which anneals specifically to the 5′ NCR of the HCV genotype 4 (HCV-4) genome.

The kits of the invention may further comprise the polymerase and an appropriate primer or set of primers. In addition, it may comprise additional reagents that are necessary for performing the invention, such as a reverse transcriptase, dNTP mixtures, buffers, molecular size standards, wax beads and the like. The reagents may be provided in pre-measured amounts so as to simplify the performance of the method. Typically, the kit may also contain detailed instructions for carrying out the method.

It will be appreciated by those skilled in the art that the sequences of the primers and probes described above can be modified without affecting their activity, i.e. their ability to act as primers or probes in the methods of the present invention. Such modified probes and primers are included in the present invention, provided that they have substantial identity with the probes mentioned above.

When comparing nucleic acid sequences for the purposes of determining the degree of homology or identity, one can use programs such as BESTFIT and GAP (both from the Wisconsin Genetics Computer Group (GCG) software package). BESTFIT, for example, compares two sequences and produces an optimal alignment of the most similar segments. GAP enables sequences to be aligned along their whole length and finds the optimal alignment by inserting spaces in either sequence as appropriate. Suitably, in the context of the present invention when discussing identity of nucleic acid sequences, the comparison is made by alignment of the sequences along their whole length.

Preferably, sequences which have substantial identity have at least 75% sequence identity and more preferably at least 90 or at least 95% sequence identity with said sequences. In some cases, the sequence identity may be 99% or above.

Desirably, the term “substantial identity” indicates that said sequence has a greater degree of identity with any of the sequences described herein than with prior art nucleic acid sequences.

Preferred features of each aspect of the invention are as for each of the other aspects mutatis mutandis. The prior art documents mentioned herein are incorporated to the fullest extent permitted by law. The abbreviations for nucleotides used herein are standard unless otherwise indicated.

The present inventors have invented a polymerase chain reaction amplification (PCR) based test that permits HCV genotype group A and HCV genotype group B to be distinguished. The test also enables detection of HCV genotypes 1, 5, 6, HCV genotype 2, HCV genotype 3, and HCV genotype 4.

The present invention will now be described further in the following examples. Reference is made to the accompanying drawings:

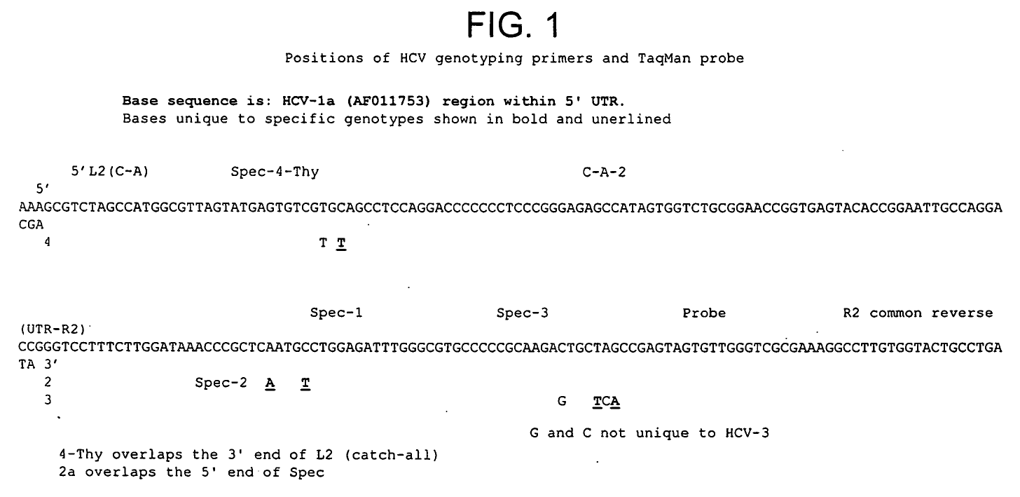

FIG. 1 illustrates the positions of the various primers and probes used in certain embodiments of the present invention within the 5′NCR of the HCV genome (SEQ ID NO: 18).

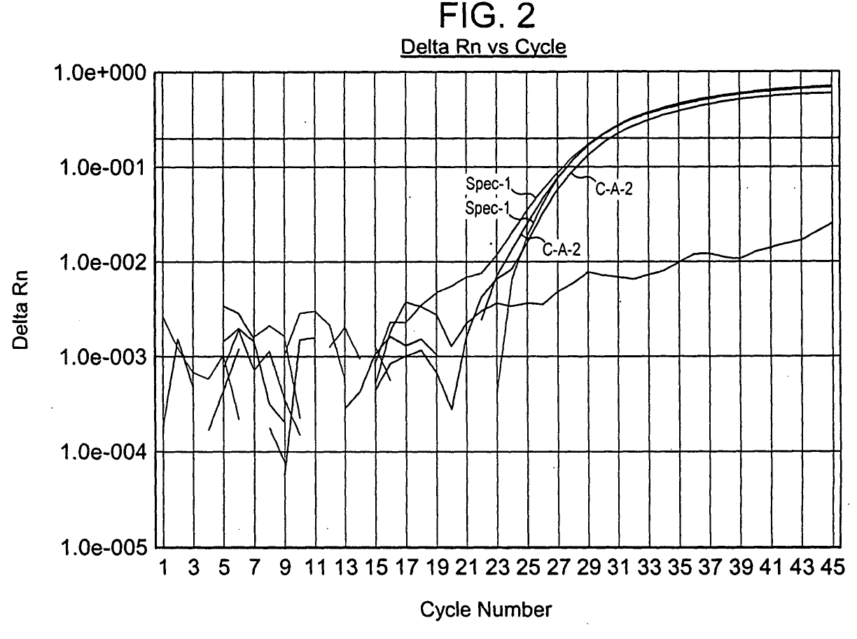

FIG. 2 illustrates the real-time amplification plot (Delta Rn vs cycle) of Example 5 with C-A-2 Catch-All primer using control RNA produced from clinical samples for HCV genotype 1.

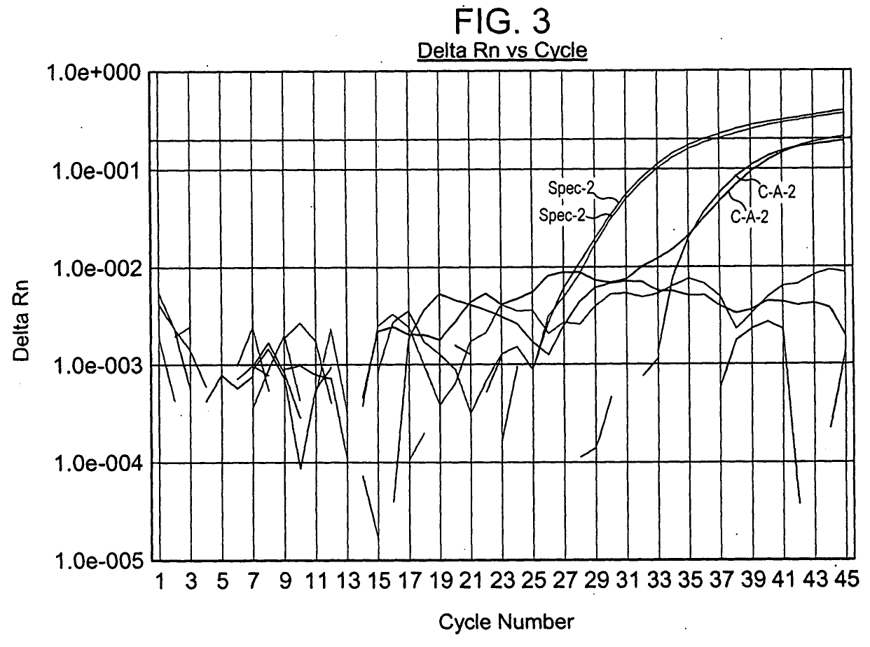

FIG. 3 illustrates the real-time amplification plot (Delta Rn vs cycle) of Example 5 with C-A-2 Catch-All primer using control RNA produced from clinical samples for HCV genotype 2.

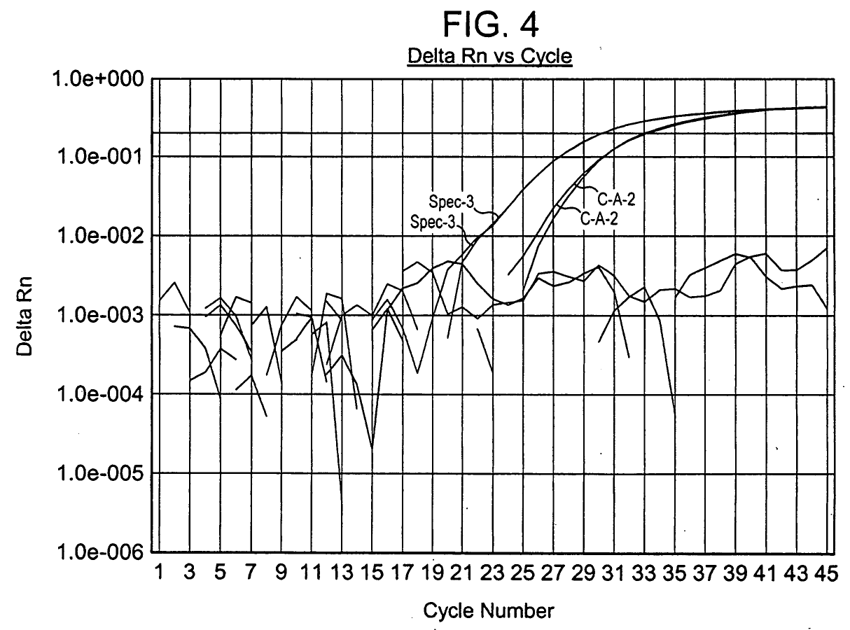

FIG. 4 illustrates the real-time amplification plot (Delta Rn vs cycle) of Example 5 with C-A-2 Catch-All primer using control RNA produced from clinical samples for HCV genotype 3.

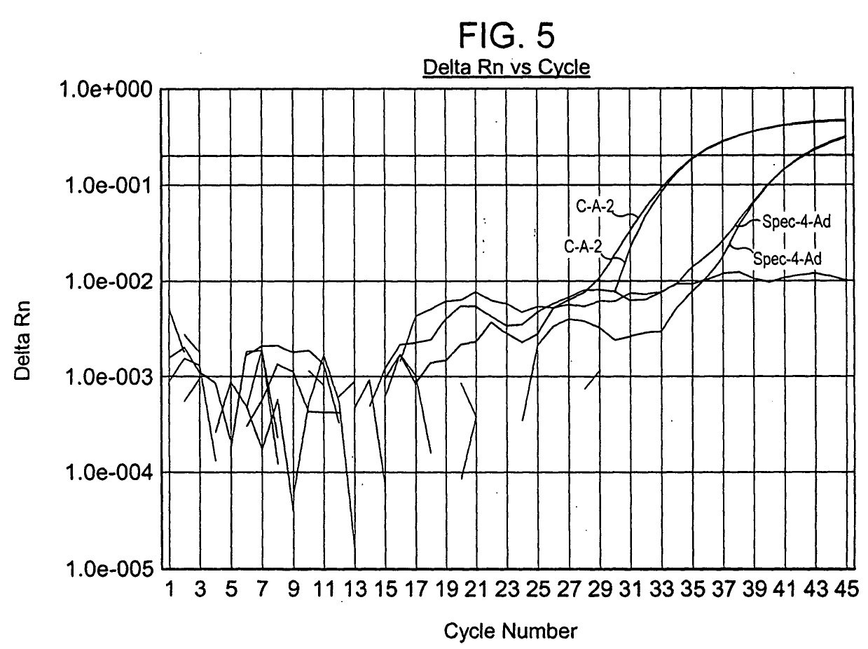

FIG. 5 illustrates the real-time amplification plot (Delta Rn vs cycle) of Example 5 with C-A-2 Catch-All primer using control RNA produced from clinical samples for HCV genotype 4.

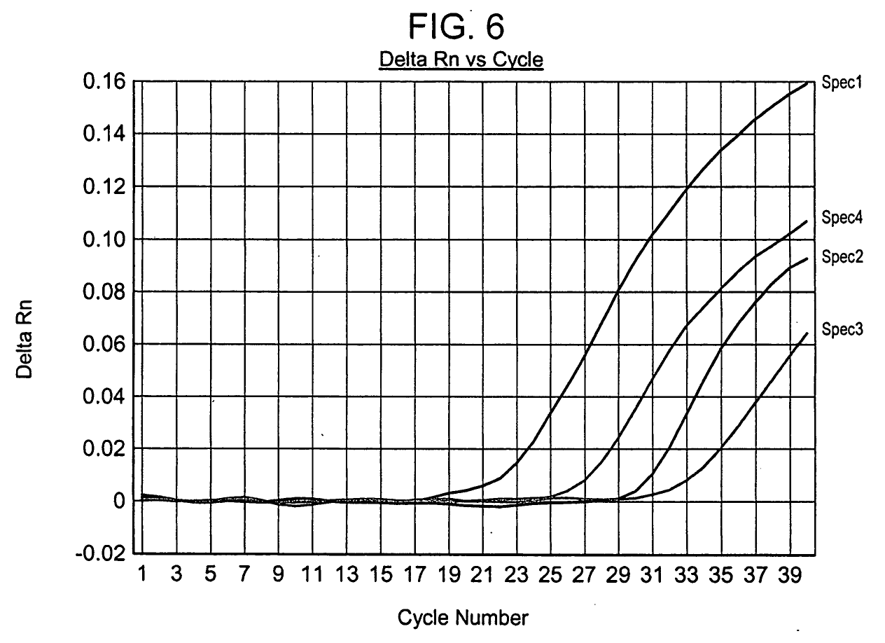

FIG. 6 illustrates the real-time amplification plot (Delta Rn vs cycle) of Example 6 using control RNA produced from clinical samples for HCV genotype 1.

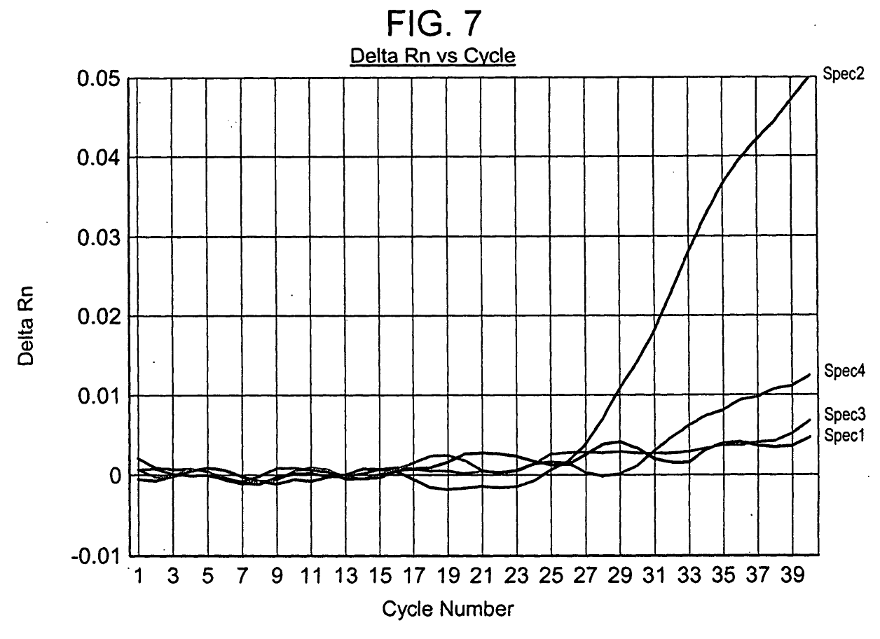

FIG. 7 illustrates the real-time amplification plot (Delta Rn vs cycle) of Example 6 using control RNA produced from clinical samples for HCV genotype 2.

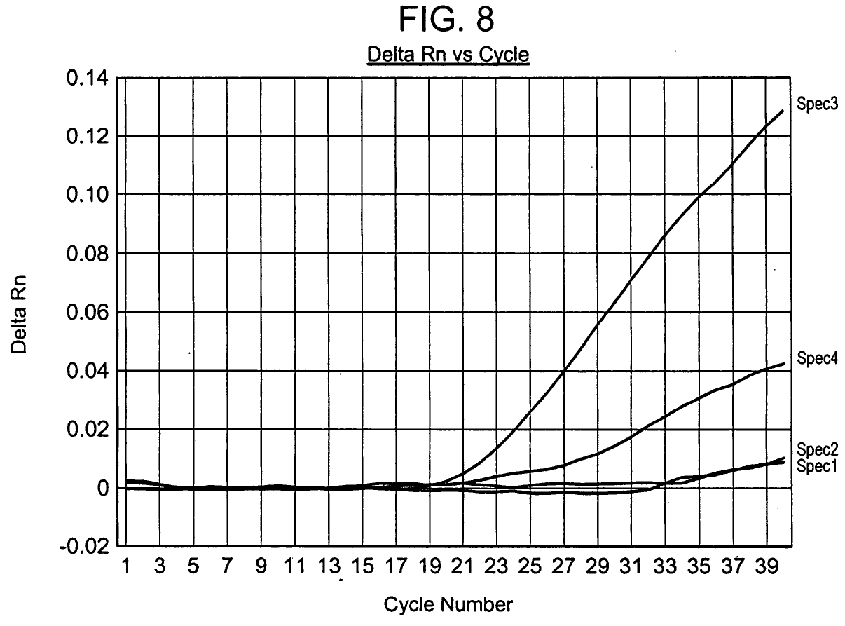

FIG. 8 illustrates the real-time amplification plot (Delta Rn vs cycle) of Example 6 using control RNA produced from clinical samples for HCV genotype 3.

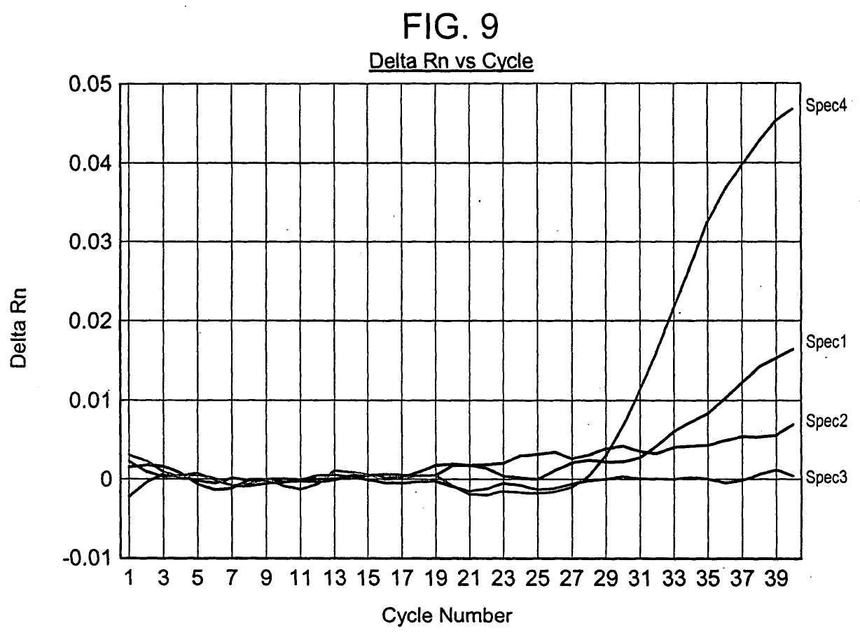

FIG. 9 illustrates the real-time amplification plot (Delta Rn vs cycle) of Example 6 using control RNA produced from clinical samples for HCV genotype 4.

### EXAMPLES

### Example 1

A genotyping assay was performed on samples of known HCV genotypes using the following sequence of primers (positions shown in FIG. 1):

Catch all (UTR-L2):

(SEQ ID NO: 1)

5′ CGI CTA GCC ATG GCG TTA G 3′
1, 5, 6 (Spec 1):

(SEQ ID NO: 6)

5′ CCG CTC AAT GCC TGG AG 3′
2 (Spec-2):

(SEQ ID NO: 7)

5′ CTT TCT TGG ATA AAC CCA CTC T 3′
3 (Spec-3):

(SEQ ID NO: 8)

5′ GTG CCC CCG CGA GAT CA 3′
4 (Spec-4-Thy)

(SEQ ID NO: 9)

5′ CCA TGG CGT TAG TAT GAG TTT T 3′
Reverse (UTR-R2):

(SEQ ID NO: 3)

5′ CAG GCA GTA CCA CAA GGC 3′
Probe (L1):

(SEQ ID NO: 11)

5′ FCG CIA CCC AAC ICT ACT IGG CTA GT 3′
(Where I = deoxyinosine, F = 6-FAM and
3′T = TAMRA-3′-phosphate)

In the following, the Tm was calculated using http://www.genosys.co.uk/technical/franmeset.html

The primers Spec-2 and Spec-3 have the following characteristics:

| | |
|---|---|
| Spec-2 5′ CTTTCTTGGATAAACCCACTCT 3′ | (SEQ ID NO: 7) |

(The A and T are unique to HCV genotype 2)

| | | | |
|---|---|---|---|
| Length | 22 | Molecular Weight | 6607.40 |
| Melting Temperature | 60.01 | Percent GC | 40.91% |
| Secondary Structure | Weak | Primer Dimer | No |

| | |
|---|---|
| Spec-3 5′ GTGCCCCCGCGAGATCA 3′ | (SEQ ID NO: 8) |

(The T and A are unique to HCV genotype 3)

| | | | |
|---|---|---|---|
| Length | 17 | Molecular Weight | 5142.40 |
| Melting Temperature | 71.24 | Percent GC | 70.59% |
| Secondary Structure | None | Primer Dimer | No |

With regard to Spec-4-Thy, primers that anneal specifically to 5′ NCR of HCV-4 genome were chosen and produced with varying lengths as follows.

| | |
|---|---|
| 4a: 5′ ATGGCGTTAGTATGAGTGTT 3′ | (SEQ ID NO: 14) |

| | | | |
|---|---|---|---|
| Length | 20 | Molecular Weight | 6207.00 |
| Melting Temperature | 56.14 | Percent GC | 40.00% |
| Secondary Structure | None | Primer Dimer | No |

| | |
|---|---|
| 4b: 5′ CATGGCGTTAGTATGAGTGTT 3′ | (SEQ ID NO: 15) |

| | | | |
|---|---|---|---|
| Length | 21 | Molecular Weight | 6495.20 |
| Melting Temperature | 59.11 | Percent GC | 42.86% |
| Secondary Structure | Very weak | Primer Dimer | No |

| | |
|---|---|
| 4c: 5′ CCATGGCGTTAGTATGAGTGTT 3′ | (SEQ ID NO: 16) |

| | | | |
|---|---|---|---|
| Length | 22 | Molecular Weight | 6783.40 |
| Melting Temperature | 62.60 | Percent GC | 45.45% |
| Secondary Structure | Very weak | Primer Dimer | No |

It should be noted that T in each of the above primers is unique to HCV genotype 4.

The initial work with the type 4 primers 4a, 4b and 4c indicated non-specific binding to other HCV genotypes (1, 3). To increase specificity, mismatches were introduced in the primer up to three bases in from the 3′ end. This reduces the affinity of binding of the primer, but as the genotype 4 has the specific (and unique) terminal T, the primers were expected to maintain the ability to bind to this sequence.

All the primers are based around the 22 mer (4c) as this has the most similar Tm to the reverse primer:

(SEQ ID NO: 16)

| | |
|---|---|
| 4c: | 5′ CCA TGG CGT TAG TAT GAG TGT T 3′ |

(SEQ ID NO: 17)

| | |
|---|---|
| 4-Ino: | 5′ CCA TGG CGT TAG TAT GAG TIT T 3′ |

An inosine (I) was substituted. This base is capable of binding to any of the four common bases, but with a reduced affinity. This may still give non-specific binding.

4-Thy (Spec-4-Thy):

(SEQ ID NO: 9)

5′ CCA TGG CGT TAG TAT GAG TTT T 3′

A thymine (T) was substituted. Thymine will not bind to the cytosine residue on the compliment with any affinity. This mismatch should strongly prevent the non-specific binding, but may affect the binding to the type 4 sequence too.

4-Ad (Spec-4-Ad):

(SEQ ID NO: 10)

5′ CCA TGG CGT TAG TAT GAG TAT T 3′

An adenine (A) was substituted in place of thymine. Adenine will bind to the cytosine residue with a small affinity. This results in better primer binding characteristic, whilst retaining specificity to HCV-4.

The specificity of binding of these primers was investigated and the performance of Spec-4-Thy was initially regarded to be higher than that of Spec-4-Ad. 4 Ino was observed to bind non-specifically to other HCV genotypes and give a TaqMan signal. Further investigations demonstrated that the 4-Ad (Spec-4-Ad) primer works more successfully than the 4-Thy (Spec-4-Thy) primer at lower viral loads (approximately 10³ IU/ml serum).

Three panels consisting of members with known HCV genotypes were tested with the above assay.

RNA was isolated and cDNA produced from samples as follows. From a 200 μl (or 500 μl) sample of serum, HCV RNA was extracted using mRNA extraction kit (such as from QIAgen Ltd, Crawley, West Sussex) as per manufacturer's instructions. 11.5 μl of RNA solution as extracted above was added to 0.5 μl of random primers (Promega UK Ltd, Southampton) in a 1.5 μl eppindorf test tube and incubated at 90° C. for 5 minutes in a heating block and then snap cooled on ice.

cDNA was then prepared by reverse transcription using the following reaction mix. 5 μl of 5× SuperScript buffer (Life Technologies (GIBCO BRL) Paisley, Scotland); 2.5 μl of 100 mM DTT; 2.5 μl of 1 mg/ml Bovine Serum Albumin acetylated (10 mg/ml) (Promega, UK Ltd, Southampton); 1.25 μl of 10 mM dNTP solution (Amersham Pharmacia, Buckinghamshire); 1.25 μl Superscript Moloney Murine Leukaemia Virus reverse transcriptase (Life Technologies (GIBCO BRL) Paisley, Scotland); 0.5 μl of RNAsin ribonuclease inhibitor (Promega, UK Ltd, Southampton).

13 μl of this solution was added to each tube containing RNA/random primers. The mix was incubated at 42° C. for 60 minutes. The reaction was stopped by heating to 95° C. for 5 minutes. The cDNA was then stored at −20° C.

The assay was then performed as follows. 1 μl of the cDNA was suspended in 20 μl of a first round reaction mix. This mix contains 1.5 mM MgCl₂, 0.2 mM deoxy nucleotide triphosphates (guanine, adenosine, threonine and cytosine) (Amersham Pharmacia, Amersham, UK), 0.1 μl of Taq polymerase (Promega UK Ltd, Southampton), 3 μM each of primers UTR-L2 and UTR-R2, and one or more of the primers Spec-1, Spec-2, Spec-3, Spec-4-Thy and the probe L1. The PCR reaction conditions were as follows: 94° C. for 1 minute for 1 cycle; 94° C. for 30 seconds; 60° C. for 20 seconds; 72° C. for 20 seconds for 50 cycles. Alternatively the PCR reaction conditions could be 50° C. for 2 minutes; 95° C. for 10 minutes; 40 to 50 cycles of 95° C. for 15 seconds and 62° C. for 1 minute.

i. Quality Control for Molecular Diagnostics (QCMD) Panel
8 member panel consisting of:
HCV-1a
HCV-1b
2×HCV-2b
HCV-3a
2×HCV-4
HCV negative

The assay was performed with primers Spec-1, Spec-2, Spec-3, Spec-4-Thy and also with the primer Spec-4-Ad. The assay correctly identified 8 out of the 8 members.

ii. Boston Biomedica Inc. Panel

10 member panel consisting of:
HCV-1a
HCV-1b
HCV-2b
HCV-3a
HCV-3b
HCV-4-h
HCV-4
HCV-5a
HCV-6a
HCV not detected

Same primers as above. The assay correctly identified all members of this panel.

iii. Teragenix Panel

25 member panel. 14 “non-HCV-1” members consisting of:
5×HCV-2b
4×HCV-3a
3×HCV-4a
HCV-5a
HCV-6a

Primers as above, but not Spec-4-Ad. The assay correctly assigned all members in this panel.

In summary, of the total number samples tested (32 (including 2 HCV not detected/negative)), 30 out of 30 HCV positive genotypes were assigned. This equates to a 100% success rate.

The accuracy of the assay is indicated in the following table:

| | N°. of samples | |
|---|---|---|
| | identified/N°. of samples | |
| HCV genotype tested | tested | Accuracy (%) |
| HCV-1 (as HCV-1, 5, 6)* | 4/4 | 100% |
| HCV-2 | 8/8 | 100% |
| HCV-3 | 7/7 | 100% |
| HCV-4 | 7/7 | 100% |
| HCV-5 (as HCV-1, 5, 6)* | 2/2 | 100% |
| HCV-6 (as HCV-1, 5, 6)* | 2/2 | 100% |
| HCV not detected/negative | 2/2 | 100% |

[*HCV-1, 5 and 6 were identified using the primer Spec-1. This primer anneals to the genomes of HCV-1, HCV-5 and HCV-6]

No false positive results occurred. Overall the assay was able to correctly identify 32 out of 32 samples. Thus, the accuracy was 100%.

### Example 2

The above assay was compared with an existing assay (Versant LiPA HCV genotyping assay) using clinical samples. The LiPA assay gives specific genotype and subtype, not a simple readout for treatment purposes or high throughput, as the LiPA technique has a greater amount of operator hands-on time and overall a longer procedure.

A total of 43 samples were run with Spec 1, Spec-2, Spec-3 and Spec-4-Thy primers indicated above. Of these samples, 40 were detected with both assays and all 40 of the samples were assigned a genotype with both assays.

The accuracy of the assay is indicated in the following table:

| | N°. of samples identified | |
|---|---|---|
| | by assay of invention/N°. | Accuracy |
| | of samples identified by | of assay of |
| HCV genotype tested | LiPA assay | invention (%) |
| HCV-1 (as HCV-1, 5, 6)* | 20/20 | 100% |
| HCV-2 | 4/4 | 100% |
| HCV-3 | 12/12 | 100% |
| HCV-4 | 2/2 | 100% |

[*HCV-1 was identified using the primer Spec-1. This primer anneals to the genomes of HCV-1, HCV-5 and HCV-6]

In addition, a co-infection of HCV-2 and HCV-4 was identified by both assays and a co-infection of HCV-1 and HCV-2 was identified by both assays. Whether the correct amplicons are being produced in the PCR was tested. After the assay reaction, 12 μl of the mix was run on 2.5% agarose gel to confirm the production of amplicons of the expected size, as indicated below:

### Catch-All (UTR-L2): 219 bp

### Spec-4-Thy: 213 bp

### Spec-1: 91 bp

### Spec-2: 103 bp

### Spec-3: 63 bp

All of the amplicons were shown to be the correct size.

### Example 3

To assess an HCV genotype 2/3 combination assay, TaqMan reactions were set up with Spec-2 and Spec-3 in equal concentrations (3 μM) as the specific forward primers. The full 8 member QCMD panel was used as in Example 1.

The TaqMan results show specificity for the combination of the primers, correctly identifying samples from HCV genotypes 2/3.

To confirm that the relevant genotypes were detected, the fragment sizes of the amplicons produced in these reactions were checked on agarose gel. The amplicons were shown to be the correct size.

### Example 4

The primers Spec-1 and Spec-4-Ad were used in combination to genotype the QCMD panel (see Example 11 above) to detect genotypes 1, 4, 5 and 6 in one reaction.

### Example 5

An alternative Catch-All primer to UTR-L2 has been designed. This Catch-All primer has been designated C-A-2 and comprises the following sequence:

5′ GAG AGC CAT AGT GGT CTG C 3′ (C-A-2) (position
133-151) (nucleotides 62-80 of SEQ ID NO: 18)

This primer has a melting temperature of 60° C. PCR using this primer with a universal HCV primer will produce an amplicon size of approximately 172 bp. This is a smaller amplicon size than that that would be obtained using UTR-L2 (see Example 2-UTR-L2 amplicon size=219 bp). Therefore, the efficiency of reaction should be increased using the C-A-2 Catch-All primer.

The position of this primer is shown in FIG. 1.

The use of the C-A-2 Catch-All primer in the assay method of the present invention was validated as follows.

### RNA Extraction

HCV RNA was extracted from infected serum using a generic commercially available kit, such as the Qiagen QIAamp® DSP Virus Kit (an IVD-D 98/79/EC approved procedure). 500 μl serum from clinical samples for HCV genotype 1, HCV genotype 2, HCV genotype 3 and HCV genotype 4 were used for this process and no modifications were made to the manufacturer's protocol. The RNA was eluted in 20 or 60 μl buffer (preferably 60 μl). It was then reverse transcribed (see below) to make cDNA or stored at −80° C.

### Reverse Transcription

A standard reverse transcriptase procedure was followed using 9 to 11.5 μl RNA (preferably 11.5 μl) as described in the previous method in Example 1 above.

However, the SuperScript and the buffer were from Invitrogen and the dNTPs were from Sigma-Aldrich.

### TaqMan Mix Components

A TaqMan reaction mix was prepared with 2.5 μl of the cDNA made above, 300 nM primers (UTR-R2, and one or two of the forward primers C-A-2, Spec-1, Spec-2, Spec-3 and Spec-4-Ad), 600 nM L1 probe and 4 mM MgCl₂, 0.2 mM dUTP, 0.1 mM dATP, dCTP and dTTP (all final concentrations), 0.25 μl UNG and 0.125 μl AmpliTaq Gold in a total volume of 25 μl. All reaction mix components were obtained from Applied Biosystems.

The PCR reaction conditions using the Applied Biosystems 7700 and 7500 programmes were as follow:

50° C.-2 minutes;
95° C.-10 minutes;
40 to 50 cycles of:
95° C.-15 seconds
62° C.-1 minute

### Results

The real-time amplification plots (of Delta Rn vs cycle) using control RNA produced from clinical samples for HCV genotype 1, HCV genotype 2, HCV genotype 3 and HCV genotype 4 are shown in FIGS. 2, 3, 4 and 5, respectively.

Analysis of the real-time amplification plots showed signals produced by each specific primer type to the corresponding HCV genotype.

In FIG. 2 for the HCV-1 sample, 2 curves with C-A-2 and 2 curves with Spec-1 were produced. The assay was negative for Spec-2, Spec-3 and Spec-4-Ad. In FIG. 3 for the HCV-2 sample, 2 curves with C-A-2 and 2 curves with Spec-2 were produced.

The assay was negative for Spec-1, Spec-3 and Spec-4-Ad. In FIG. 4 for the HCV-3 sample, 2 curves with C-A-2 and 2 curves with Spec-3 were produced. The assay was negative for Spec-1, Spec-2 and Spec-4-Ad. In FIG. 5 for the HCV-4 sample, 2 curves with C-A-2 and 2 curves with Spec-4-Ad were produced. The assay was negative for Spec-1, Spec-2 and Spec-3

These plots clearly demonstrate that an assay using the C-A-2 Catch-All primer is successful in detecting HCV genotype 1, HCV genotype 2, HCV genotype 3 and HCV genotype 4.

### Example 6

A one step amplification reaction was designed in which the reagents for reverse transcription and PCR were provided in a single tube such that both reactions were performed in a PCR machine.

For initial validation, a commercially available one-step RT-PCR kit was obtained from Qiagen (QuantiTect Probe RT-PCR Kit). Any suitable reverse transcriptase and Taq DNA polymerase combination would also be appropriate for use in the amplification reaction and are available from other suppliers.

The QuantiTect Probe RT-PCR Kit contains a mixture of two reverse transcriptase enzymes; Omniscript (designed for reverse transcription of large amounts of RNA, >50 ng) and Sensiscript (optimised for reverse transcription of small amounts of RNA, <50 ng).

The Taq DNA polymerase present in the kit is a recombinant, modified form of Taq cloned into E. coli called HotStarTaq DNA Polymerase.

All the above enzymes were added at the start of the procedure to purified viral HCV RNA in an optimised buffer.

All reactions contain the L1 Probe and UTR-R2 reverse primer in addition to one of the following:
Catch-All UTR-L2
Redesigned Catch-All: C-A-2 (see Example 5)
Spec-1 (HCV genotype 1)
Spec-2 (HCV genotype 2)
Spec-3 (HCV genotype 3)
Spec-4-Ad (HCV genotype 4)

The reaction mix was as follows:

| | Volume and final |
|---|---|
| | concentration per |
| Component | reaction (μl) |
| QuantiTect Probe RT-PCR Master | 12.5 |
| Mix | |
| Forward primer (Catch-All or | 0.5 (1 μM final concentration) |
| genotype specific) 50 μM stock | |
| Reverse primer (UTR-R2) 50 μM | 0.8 (1 μM final concentration) |
| stock | |
| TaqMan probe (L1) 20 μM stock | 1 (0.5 μM final concentration) |
| QuantiTect RT mix | 0.25 |
| RNA | 10.25 |
| Total | 25 |

After the addition of all the components, the reaction mix was placed in a PCR machine (both the Applied Biosystems 7500 and 7700 sequence detection systems were used) with the following thermo-cycle programme:

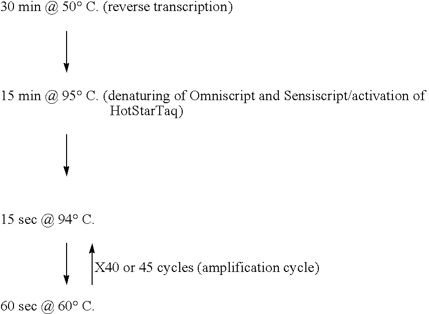

### Results

The real-time amplification plots (of Delta Rn vs cycle) using control RNA produced from clinical samples for HCV genotype 1, HCV genotype 2, HCV genotype 3 and HCV genotype 4 are shown in FIGS. 6, 7, 8 and 9, respectively.

Analysis of the real-time amplification plots showed signals produced by each specific primer type to the corresponding HCV genotype.

The impact of non-specific binding on the signal strength may be able to be reduced by increasing the amount of RNA in the reaction mix or by increasing the amount of cDNA produced.

The one-step method works as a modification to the HCV genotyping assays and reduces both the hands-on time for the operator and the overall time to result.

1. A method of determining whether HCV that is present in a sample belongs to HCV genotype group A or HCV genotype group B, comprising:
(a1) (i) subjecting the sample to a first amplification reaction using at least one primer which anneals specifically to the 5′ noncoding region (5′ NCR) of the HCV-2 genome; and
(ii) subjecting the sample to a second amplification reaction using at least one primer which anneals specifically to the 5′ NCR of the HCV-3 genome; or
(a2) subjecting the sample to an amplification reaction using at least one primer which anneals specifically to the 5′ NCR of the HCV-2 genome, and at least one primer which anneals specifically to the 5′ NCR of the HCV-3 genome; and
(b) detecting the products of the amplification reaction(s),
wherein, if a product is detected in step (a1) or (a2), the HCV is in group B and, if no product is detected in step (a1) or (a2), the HCV is in group A.

2. A method as claimed in claim 1, further comprising at least one of the steps of:
(c) subjecting the sample to an amplification reaction using at least one primer which anneals specifically to the 5′ noncoding region (5′ NCR) of the genomes of HCV-1, HCV-5 and HCV-6; and
(d) subjecting the sample to an amplification reaction using at least one primer which anneals specifically to the 5′ NCR of the HCV-4 genome; and further comprising the step of:
(e) detecting the products of the amplification reactions,
wherein, if a product is detected in at least one of step (c) and (d), the HCV is in HCV group A and, if no product is detected in at least one of step (c) and (d), the HCV is in HCV group B.

3. A method for determining whether HCV that is present in a sample belongs to HCV genotype group A or HCV genotype group B, comprising:
(a1) (i) subjecting the sample to a first amplification reaction using at least one primer which anneals specifically to the 5′ noncoding region (5′ NCR) of the genomes of HCV-1, HCV-5 and HCV-6; and
(ii) subjecting the sample to a second amplification reaction using at least one primer which anneals specifically to the 5′ NCR of the HCV-4 genome; or
(a2) subjecting the sample to an amplification reaction using at least one primer which anneals specifically to the 5′ NCR of the genomes of HCV-1, HCV-5 and HCV-6, and at least one primer which anneals specifically to the 5′ NCR of the HCV-4 genome; and
(b) detecting the products of the amplification reactions,
wherein, if a product is detected in step (a1) or step (a2), the HCV is in group A and, if no product is detected in step (a1) or step (a2), the HCV is in group B.

4. A method as claimed in claim 3, further comprising at least one of the steps of:
(c) subjecting the sample to an amplification reaction using at least one primer which anneals specifically to the 5′ noncoding region (5′ NCR) of the HCV-2 genome; and
(d) subjecting the sample to an amplification reaction using at least one primer which anneals specifically to the 5′ NCR of the HCV-3 genome; and further comprising the step of
(e) detecting the products of the amplification reactions,
wherein, if a product is detected in at least one of step (c) and (d), the HCV is in HCV group B and, if no product is detected in at least one of step (c) and (d), the HCV is in HCV group A.

5. A method of detecting HCV genotype 1, 5 or 6 (HCV-1, HCV-5 or HCV-6), HCV genotype 2 (HCV-2), HCV genotype 3 (HCV-3), or HCV genotype 4 (HCV-4) in a sample, comprising:
(a1) (i) subjecting the sample to an amplification reaction using at least one primer which anneals specifically to the 5′ noncoding region (5′ NCR) of the genomes of HCV-1, HCV-5 and HCV-6; and
(ii) subjecting the sample to an amplification reaction using at least one primer which anneals specifically to the 5′ NCR of the HCV-2 genome; and
(iii) subjecting the sample to an amplification reaction using at least one primer which anneals specifically to the 5′ NCR of the HCV-3 genome; and
(iv) subjecting the sample to an amplification reaction using at least one primer which anneals specifically to the 5′ NCR of the HCV-4 genome; or
(a2) subjecting the sample to an amplification reaction using at least one primer which anneals specifically to the 5′ NCR of the genomes of HCV-1, HCV-5 and HCV-6, at least one primer which anneals specifically to the 5′ NCR of the HCV-2 genome; at least one primer which anneals specifically to the 5′ NCR of the HCV-3 genome; and at least one primer which anneals specifically to the 5′ NCR of the HCV-4 genome; and
(c) detecting the products of the amplification reactions
to thereby detect HCV genotype 1, 5 or 6 (HCV-1, HCV-5 or HCV-6), HCV genotype 2 (HCV-2), HCV genotype 3 (HCV-3), or HCV genotype 4 (HCV-4) in a sample.

6. A method as claimed in any one of the preceding claims, further comprising subjecting the sample to a preliminary amplification reaction to detect whether any HCV genotype is present in the sample using primers which anneal to a region of the 5′ NCR which is conserved among all HCV genotypes.

7. A method as claimed in claim 6, wherein the primers which anneal to a region of the 5′ NCR which is conserved among all HCV genotypes have the following sequences:
Forward:
(SEQ ID NO: 1)

| | |
|---|---|
| i) | 5′ CGI CTA GCC ATG GCG TTA G 3′ (UTR-L2) |
| | (position 76-94): |
| or | |

(SEQ ID NO: 2)

| | |
|---|---|
| ii) | 5′ GAG AGC CAT AGT GGT CTG C 3′ (C-A-2) |
| | (position 133-151): |

Reverse:
(SEQ ID NO: 3)
5′ CAG GCA GTA CCA CAA GGC 3′ (UTR-R2):
(position 295-278)

8. A method as claimed in any one of claims 1-5, further comprising subjecting the sample to a further preliminary amplification reaction to isolate HCV material using primers universal for all HCV genotypes.

9. A method as claimed in claim 8, wherein the primers universal for all HCV genotypes comprise the following sequences:
Forward:
(SEQ ID NO: 4)
5′ GGA ACT WCT GTC TTC ACG C 3′ (UTR-R1):
Reverse:
(SEQ ID NO: 5)
5′ ACG GTC TAC GAG ACC TC 3′ (UTR-R1):

10. A method as claimed in any one of claims 2, 3, 4 or 5, wherein the at least one primer which anneals specifically to the 5′ NCR of the genomes of HCV-1, HCV-5 and HCV-6 comprises the sequence:
(SEQ ID NO: 6)
5′ CCG CTC AAT GCC TGG AG 3′ (Spec-1).

11. A method as claimed in any one of claims 1, 2, 4 or 5, or any one of claims 6 to 10 when dependent on claim 1, 2, 4 or 5, wherein the at least one primer which anneals specifically to the 5′ NCR of the HCV-2 genome comprises the sequence:
(SEQ ID NO: 7)
5′ CTT TCT TGG ATA AAC CCA CTC T 3′ (Spec-2).

12. A method as claimed in any one of claims 1, 2, 4 or 5, or any one of claims 6 to 11 when dependent on claim 1, 2, 4 or 5, wherein the at least one primer which anneals specifically to the 5′ NCR of the HCV-3 genome comprises the sequence:
(SEQ ID NO: 8)
5′ GTG CCC CCG CGA GAT CA 3′ (Spec-3).

13. A method as claimed in any one of claims 2, 3 or 5, or any one of claims 4, 6 to 12 when dependent on claim 2, 3 or 5, wherein the at least one primer which anneals specifically to the 5′ NCR of the HCV-4 genome comprises a sequence selected from:
(SEQ ID NO: 9)
5′ CCA TGG CGT TAG TAT GAG TTT T 3′ (Spec-4-Thy).
(SEQ ID NO: 10)
5′ CCA TGG CGT TAG TAT GAG TAT T 3′ (Spec-4-Ad).

14. A method as claimed in any one of claims 10 to 13, wherein the at least one primer which anneals specifically to the 5′ NCR of the genome of HCV-1, HCV-5 or HCV-6, HCV-2, HCV-3 or HCV-4 respectively is a forward primer and the reverse primer comprises the sequence:
(SEQ ID NO: 3)
5′ CAG GCA GTA CCA CAA GGC 3′ (UTR-R2).

15-17. (canceled)

18. A method as claimed in any one of claims 1-5, wherein the amplification reaction is the polymerase chain reaction (PCR) or reverse transcriptase polymerase chain reaction (RT-PCR).

19. A method as claimed in claim 18, wherein the amplification reaction comprises:
a) raising the temperature of a reaction mix comprising HCV RNA, reverse transcriptase, DNA polymerase and primers, to activate the reverse transcriptase; and
b) raising the temperature further to inactivate the reverse transcriptase and to activate the DNA polymerase.

20. A method as claimed in any one of claims 1-5, wherein detection of the product of the, or each, amplification reaction is by agarose gel electrophoresis.

21. A method as claimed in any one of claims 1 to 5, wherein detection of the product of the, or each, amplification reaction is by fluorescent analysis in which amplification of HCV specific nucleic acid causes fluorescence of a probe.

22. A method as claimed in claim 21, wherein the probe comprises the sequence:
(SEQ ID NO: 11)
5′ FCG CIA CCC AAC ICT ACT IGG CTA GT 3′ (L1).
where F = 6-FAM, 3′-T + TAMRA.

23. A method as claimed in any one of claims 1 to 5, wherein detection of the product of the, or each, amplification reaction is by one or more molecular beacon primers.

24. A method as claimed in claim 23, wherein the molecular beacon primer comprises the sequence:
(SEQ ID NO: 12)
5′ FCA CCT TCA CCC TCA GAA GGM GCC GCT CAA TGC CTG
GAG 3′.
(F = FAM; M = MeREDdU and U = Uraci1) (MBP-LR-1.

25. A method as claimed in claim 24, wherein the molecular beacon primer is a forward primer and the reverse primer comprises the sequence:
(SEQ ID NO: 3)
5′ CAG GCA GTA CCA CAA GGC 3′ (UTR-R2).

26. A method as claimed in claim 23, wherein an additional amplification is performed using at least one molecular beacon primer which is universal for all HCV genotypes.

27. A method as claimed in claim 26, wherein the molecular beacon primer comprises the sequence:
Forward:
(SEQ ID NO: 8)
5′ FCA CCT TCA CCC TCA GAA GGM GCG UCT AGC CAT GGC
GTT AG 3′.
(F = FAM; M = MeREDdU and U = Uraci1) MBP-LR-ALL

28. A method as claimed in claim 27, wherein the molecular beacon primer is a forward primer and the reverse primer comprises the sequence:
(SEQ ID NO: 3)
5′ CAG GCA GTA CCA CAA GGC 3′ (UTR-R2).

29. A kit for determining whether HCV that is present in a sample belongs to HCV genotype group A or HCV genotype group B, comprising:
(a) at least one primer which anneals specifically to the 5′ NCR of the HCV genotype 2 (HCV-2) genome; and
(b) at least one primer which anneals specifically to the 5′ NCR of the HCV genotype 3 (HCV-3) genome.

30. A kit as claimed in claim 29, wherein the kit further comprises:
(c) at least one primer which anneals specifically to the 5′ NCR of the HCV genotype 4 (HCV-4) genome; and
(d) at least one primer which anneals specifically to the 5′ NCR of the genomes of HCV-1, HCV-5 and HCV-6.

31. A kit for determining whether HCV that is present in a sample belongs to HCV genotype group A or HCV genotype group B, comprising:
(a) at least one primer which anneals specifically to the 5′ NCR of the genomes of HCV genotype 1, 5 and 6; and
(b) at least one primer which anneals specifically to the 5′ NCR of the HCV genotype 4 (HCV-4) genome.

32-34. (canceled)

35. A kit for determining whether HCV that is present in a sample belongs to HCV genotype group A or HCV genotype group B, comprising:
(a) at least one primer which anneals specifically to the 5′ NCR of the HCV genotype 2 (HCV-2) genome; and
(b) at least one primer which anneals specifically to the 5′ NCR of the HCV genotype 3 (HCV-3) genome; and optionally further or alternatively comprising
(c) at least one primer which anneals specifically to the 5′ NCR of the HCV genotype 4 (HCV-4) genome; and
(d) at least one primer which anneals specifically to the 5′ NCR of the genomes of HCV-1, HCV-5 and HCV-6.

36. A nucleotide molecule suitable for use in an amplification reaction comprising one of the following sequences:
(SEQ ID NO: 6)
5′ CCG CTC AAT GCC TGG AG 3′ (Spec-1).
(SEQ ID NO: 7)
5′ CTT TCT TGG ATA AAC CCA CTC T 3′ (Spec-2).
(SEQ ID NO: 8)
5′ GTG CCC CCG CGA GAT CA 3′ (Spec-3).
(SEQ ID NO: 9)
5′ CCA TGG CGT TAG TAT GAG TTT T 3′ (Spec-4-Thy)
(SEQ ID NO: 10)
5′ CCA TGG CGT TAG TAT GAG TAT T 3′ (Spec-4-Ad)
(SEQ ID NO: 1)
5′ CGI CTA GCC ATG GCG TTA G 3′ (UTR-L2)
(SEQ ID NO: 2)
5′ GAG AGC CAT AGT GGT CTG C 3′ (C-A-2)
(SEQ ID NO: 3)
5′ CAG GCA GTA CCA CAA GGC 3′ (UTR-R2)
(SEQ ID NO: 19)
5′ GGA ACT TCT GTC TTC ACG C 3′ (UTR-L1)

37. A pair of primers comprising a nucleic acid molecule having the following nucleotide sequence:
Forward:
(SEQ ID NO: 6)
5′ CCG CTC AAT GCC TGG AG 3′ (Spec-1).
Reverse:
(SEQ ID NO: 3)
5′ CAG GCA GTA CCA CAA GGC 3′ (UTR-R2)
Forward:
(SEQ ID NO: 7)
5′ CTT TCT TGG ATA AAC CCA CTC T 3′ (Spec-2).
Reverse:
(SEQ ID NO: 3)
5′ CAG GCA GTA CCA CAA GGC 3′ (UTR-R2)
Forward:
(SEQ ID NO: 8)
5′ GTG CCC CCG CGA GAT CA 3′ (Spec-3).
Reverse:
(SEQ ID NO: 3)
5′ CAG GCA GTA CCA CAA GGC 3′ (UTR-R2)
Forward:
(SEQ ID NO: 9)
5′ CCA TGG CGT TAG TAT GAG TTT T 3′ (Spec-4 -Thy)
Reverse
(SEQ ID NO: 3)
5′ CAG GCA GTA CCA CAA GGC 3′ (UTR-R2)
Forward:
(SEQ ID NO: 10)
5′ CCA TGG CGT TAG TAT GAG TAT T 3′ (Spec-4-Ad)
Reverse:
(SEQ ID NO: 3)
5′ CAG GCA GTA CCA CAA GGC 3′ (UTR-R2)
